(19) United States
(12) Patent Application Publication
(10) Pub. No.: US 2002/0065542 A1
(43) Pub. Date: May 30, 2002

(54) METHOD AND APPARATUS FOR TREATING AN ANEURYSM

(76) Inventors: RONALD G. LAX, PALM CITY, FL (US); STUART D. EDWARDS, PORTOLA VALLEY, CA (US)

Correspondence Address: RYAN KROMHOLZ & MANION, S.C., P.O. BOX 26618, MILWAUKEE, WI, 53226-0618, US

(21) Appl. No.: 09/296,855

(22) Filed: Apr. 22, 1999

Related U.S. Application Data
(60) Provisional application No. 60/082,711, filed on Apr. 22, 1998.

(51) Int. Cl. A61F 7/00; A61F 7/12

(52) U.S. Cl. 607/99; 607/113

(57) ABSTRACT

Embodiments of this invention provide for an apparatus for treating an aneurysm in a vessel. The apparatus includes an insertion member adapted to be inserted into a lumen of the vessel. A first electrode device is coupled to the insertion member and applies electrical energy to an interior surface of the vessel to shrink the aneurysm at an interior surface that is part of a surface that circumscribes a cross-section of the vessel.

### CROSS REFERENCE TO RELATED APPLICATION

This application claims the benefit of prior filed copending Provisional Application No. 60/082,711, filed Apr. 22, 1998, [Attorney Docket No. 18259.712], entitled Method and Apparatus For Treating An Aneurysm. This application also incorporates by reference (first incorporated by reference in Provisional Application No. 60/082,711) U.S. Application No. 08/984,481 entitled Method of Forming a Stent In Situ, filed Dec. 1, 1997.

### BACKGROUND OF THE INVENTION

Referring to FIG. 1, an early stage abdominal aortic aneurism 1 (AAA) is shown. Renal vessels that go to the heart are connected to the aortic artery 2 at the top; at the bottom, the aortic artery divides at a bifurcation 3. The aneurism 1 can expand up to the renal arteries and down to the bifurcation.

Referring to FIG. 2, a more advanced stage AAA is shown. If a given section of a lumen has a certain wall thickness of X when not affected by an aneurysm, then the wall thickness of the section is fractionally less, for instance 0.5×, when that portion of the lumen is affected by an aneurysm. This result stems from the lumen wall thinning to compensate for its cross-section increasing.

Typically with an aneurism 1, one approach has been to do open surgery, put in a device, and close it up (i.e., take the excess out and stitch it up). In more detail, the present procedure includes having a surgeon go in, open the patient up, cut the aneurism open, and put in an implant which can include Dacron, or a braided hose. The surgeon will then suture it in. In addition, the surgeon will cut the excess and wrap it around, leaving excess tissue; the surgeon will stitch it right down the seam.

One of the problems is that the reconfigured wall is already thinned and any continued expansion remains problematic. Of course, there is still blood pressure and the patient may experience continuing distention, therefore, this prior art approach is often not a permanent fix.

In the past, grafts and/or stents have also been used. However, these approaches also have problems.

What is needed is an approach to treating aneurysms that avoids the problems inherent to the prior art described above. Heretofore, this need has not been met.

### SUMMARY OF THE INVENTION

Embodiments of this invention provide for an apparatus for treating an aneurysm in a vessel. The apparatus includes an insertion member adapted to be inserted into a lumen of the vessel. A first electrode device is coupled to the insertion member and applies electrical energy to an interior surface of the vessel to shrink the aneurysm at an interior surface that is part of a surface that circumscribes a cross-section of the vessel.

In a variation, the electrode rotates about the insertion member to heat the interior surface of the vessel with electrical energy. Alternatively, the electrode may be shaped, such as in the form of a cylinder, to apply energy to the interior of the vessel.

In another embodiment, the first electrode device includes an inner balloon positioned over the insertion member, and an outer balloon positioned over the inner balloon to be proximal to at least a portion of the interior surface. A plurality of electrodes form a pattern on an outer surface of the outer balloon and apply the electrical energy to the interior surface.

Another embodiment of the invention provides that the first electrode device includes an inelastic inner balloon positioned over the insertion member, and an elastic outer balloon surrounding at least a portion of the inner balloon to be near the interior surface. A plurality of electrodes form a pattern on an outer surface of the outer balloon to apply electrical energy to the interior surface.

### BRIEF DESCRIPTION OF THE DRAWINGS

FIG. 1 illustrates a schematic view of an abdominal aortic aneurism (AAA) at an early stage.

FIG. 2 illustrates a schematic view of an abdominal aortic aneurism (AAA) at a more advanced stage.

FIG. 3 illustrates a schematic view of an aneurism being treated by a rotating and/or linear traversing electrode, representing an embodiment of the invention.

FIG. 4 illustrates a schematic view of a dual balloon catheter with thermocouples and pressure transducers, representing an embodiment of the invention.

FIG. 5 illustrates a schematic view of an aneurism at various treatment stages (i.e., before treatment, after shrinkage, and after foam deployment), representing an embodiment of the invention.

FIG. 6 illustrates a schematic view of a dual balloon catheter with thermocouples, pressure transducers, and an electrolyte solution, representing an embodiment of the invention.

### DETAILED DESCRIPTION OF THE DRAWINGS

Referring to FIG. 3, an apparatus 100 for treating an aneurysm under an embodiment of this invention is shown inserted into a vessel 110 having an affected aneurysm portion 50. The apparatus 100 heats an interior surface 12 lining a lumen 14 of the vessel 110 at the affected portion 50. Heating the interior surface 12 at the affected portion 50 treats the aneurysm by causing the vessel 110 to shrink. In addition, heating the interior surface 12 may cause scar tissue to form inside the vessel 10 that strengthens portions of the vessel 110 that have deteriorated as a result of the aneurysm. As a result, the apparatus 100 is more efficient and effective in treating aneurysms than previous known devices that require excess tissue to be cut-away.

The apparatus 100 includes an electrode device 20 and an insertion member 50. The insertion member 10 inserts into the lumen 14 of the vessel 110. Preferably, the insertion member 10 is a catheter that guides, moves, or rotates the electrode device 20 within the lumen 14.

In an embodiment such as shown by FIG. 3, the electrode device 20 includes a barrel-shaped working electrode. The electrode device 20 heats the interior surface 12 by rotating about an axis 25 defined within the lumen. The electrode device 20 can supply heat to the interior surface 12 by, for example, incorporating a radio frequency powered electrode. Another electrode (e.g., an electrode patch) can be placed someplace on the body, for example, on the back or shoulders, or some other big muscles. The radio frequency current (preferably approximately 480 to 490 KHz frequency) heats the tissue near the working electrode. The invention can operate at fairly low wattage, for example, from approximately 3 watts to approximately 15 or 20 watts, particularly in treating tissue where there is a high content of collagen and elastin. This heating will allow the vessel tissue that had deteriorated as a result of the aneurysm to density and will allow the fibrils to close. This embodiment of the invention can shrink the aneurysm portion of the vessel 110 by approximately 50% in volume.

The voltage differential between the working electrode and the other electrode can be approximately 200 volts. What is more important is the current density. The other electrode can be termed an indifferent electrode and is located away from the tissue to be heated and will have a surface area that is smaller than the working electrode. The indifferent electrode may have an area of approximately 24 sq. inches. By using a working electrode with a smaller diameter, the watts per square centimeter increases, compared to the indifferent electrode. The indifferent electrode is termed indifferent because its current is so spread out there is no heating. The working electrode contact area may be approximately 0.10 cm². Therefore, the wattage density at the working electrode is much higher than at the indifferent electrode.

The temperature range to achieve shrinkage is approximately 45 to approximately 95 degrees centigrade. An optimum temperature range is from approximately 60 to approximately 80, and preferably approximately 60 to approximately 75 degrees centigrade.

Still referring to FIG. 3, as the electrode device 20 is preferably rotated around the circumference of the lumen 14 to heat the interior surface 12 circumscribing the cross-section of the affected portion 50. This results in sizably shrinking the vessel 110. The rotation can be continuous or in discrete steps. In the case of the embodiment depicted in FIG. 3, the electrode device 20, or an electrode of the electrode device 20, can be moved longitudinally along the axis defined within the lumen, either continuously or in discrete steps.

In another embodiment, the electrode device 20 may include a shoe-shaped electrode. This embodiment would include, a probe and a metallic electrode. In other embodiments, an electrode of the electrode device 20 may be cylindrical and symmetric, thereby obviating the need for any rotational movement during shrinkage.

Embodiments of the invention preferably employ a catheter as the insertion member 10 to deploy the electrode device 20. This provides the apparatus 100 with the ability to insert, move, and remove, the electrode device 20, or electrodes of the electrode device 20. This mobility is especially advantageous with regard to the vascular system.

Moreover, by watching the shrinkage under a fluoroscope, a surgeon can see the outline of the lumen as it is treated. The surgeon can see the aneurism and will be able to monitor the shrinkage. Ideally, the surgeon may want to shrink the tissue until the lumen 14 assumes a near-normal shape due to the movement of the densified tissue. Alternatively, a surgeon may want to shrink until a wall of the lumen 14 has been significantly thickened, particularly if a near-normal shape is not obtainable.

Shrinking the vessel 110 in this manner may create some scar tissue. The scar tissue toughens the lumen 12, which is a desired effect because an aneurism can be life threatening. With the invention, a surgeon can thicken the lumen tissue by heat mediated therapy on the interior surface 12 of the affected portion 50, thereby simultaneously causing shrinkage and contraction of the vessel 10 while toughening and thickening the walls of the vessel. Consequently, a surgeon using the invention may obviate the need to do a graft, or even a stent.

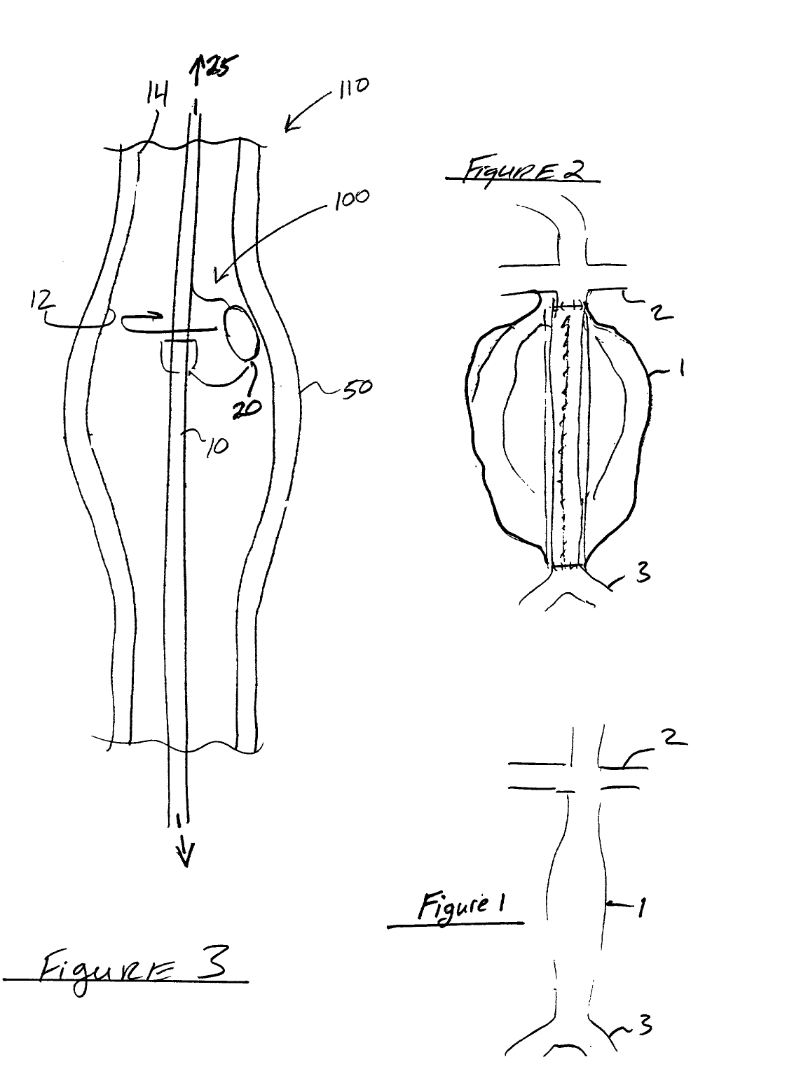

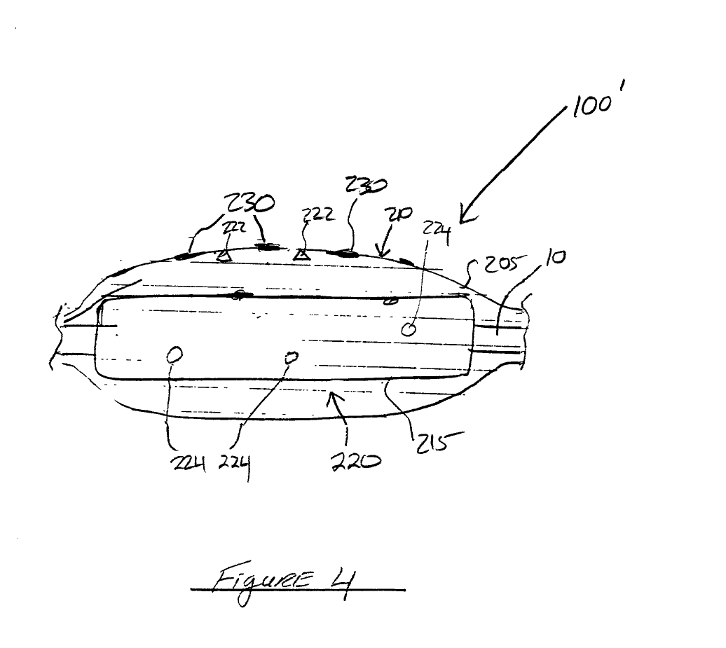

The treatment described above is effective, and relatively easy for a patient to withstand. As a result, another advantage of the invention is that it encourages and facilitates early detection of aneurysms such as the abdominal aortic aneurysms described above. Earlier detection allows a physician to treat the aneurysm when it has the appearance depicted in FIG. 1. Early stage treatment generally results in aneurysms that are more easily treated with RF energy, as described above.

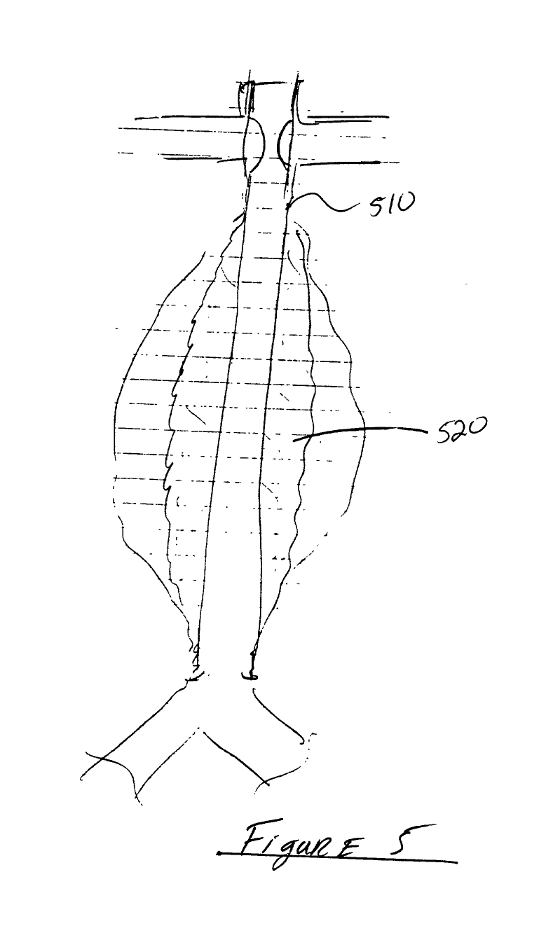

In embodiments of this invention, the electrode device 20 can include a roller electrode that walks around the inside surface 12 of the affected portion 50, as shown by FIG. 3. Alternatively, embodiments of the invention can be based on a balloon that has a series of electrodes on the outside, as depicted in FIGS. 4 and 6.

FIG. 4 illustrates an alternative apparatus 100′ for treating aneurysms under this invention. The apparatus 100′ includes an electrode device that incorporates a dual balloon configuration that surrounds the insertion member 10 within the lumen 12 of the affected portion 50. The dual balloon configuration includes an outer balloon 210 and an inner balloon 220. Preferably, the outer balloon 210 is elastic, and the inner balloon 210 is expandable but non-elastic. A series or pattern of electrodes 230 may be disbursed on an outside surface 205 of the outer balloon 210. One or more thermocouples 222 can be located on the outer surface 205 of the outer (and/or the inner) balloon 210. In addition, one or more pressure transducers or sensors 224 can be dispersed over an outer surface 215 of the inner balloon 220. This configuration creates a feedback balloon probe that paints the interior surface 12 in the affected portion 50 of the vessel 110.

In one embodiment, the electrodes 230 could be patterned on the outer surface 205 of the outer balloon (or the inner balloon), and then modulated in a predetermined sequence. The modulation may simulate an electrode such as a barrel electrode rotating about a longitudinal axis to heat the interior surface 12, such as in a manner described with FIG. 3. In much the same way a series of bit lines can be used to address a particular cell on a microchip, a series of electrode selector circuits (not shown) could be used to define a sequence of spots on either the outer or inner balloon. This would create a device that could spatially modulate the shrinkage (“walk around”), without moving any parts.

FIG. 6 illustrates a variation to the embodiment described with FIG. 4. The inner nonelastic balloon 220 of the apparatus 100′ is expandable to a diameter of interest (e.g., the diameter that the interior of a lumen is intended to have after treatment). An outer elastic balloon 210 is expandable to the diameter of the interior of the affected portion 50. The resulting configuration forms a distal occluding balloon and a proximal occluding balloon. The space between the inner inelastic balloon 220 and the outer elastic balloon 210 may be filled with a saline electrolyte that is pumped in.

In the embodiment of FIG. 6, the outer balloon 210 is highly elastic in nature (a silicon balloon, for instance), and the inner balloon 220 is inelastic. The outer balloon 210 preferably surrounds the inner balloon 220 in its entirety, but alternatively surrounds only a portion of the inner balloon 220. In an embodiment, the inner balloon 220 is made of a material like PET, and may include a single outer diameter, or multiple outer diameters. The inner balloon 220 can be inflated with a working fluid (saline) that expands it to a predefined diameter of interest. A pair of damming balloons 260, 262 can be used to contain the working fluid within the inner balloon 220.

The outer balloon 210 can be inflated until it contacts the inner wall(s) of the lumen 12. Contact of the outer balloon 210 with the inner wall(s) could be ascertained by means of pressure transducers or sensors 224 located on the outer balloon 210, and preferably, on the outer surface 205 of the outer balloon 210. A plurality of pressure sensors 224 are also located on the outer surface 215 of the inner balloon 220. In this way, most (or all) of the electrodes 230 on the outer surface 205 of the outer balloon 210 would be known to be in contact with the interior surface of the affected portion 50 of the vessel 110. In this configuration, the interior surface 12 can be heated by powering the electrodes 230.

In one embodiment, the power to the electrodes 230 is multiplexed with circuitry so that a dynamic hot zone can be rotated or walked around the outer surface 205 of the outer balloon 210. Various areas of tissue surrounding the outer balloon 210 may then be heated in a sequence, either continuously or in discrete steps. In this embodiment, the outer and/or inner balloons 210 and 220 can also be equipped with thermocouples 222. The thermocouples 222 can give temperature feedback so that when a desired temperature is reached, the power to the electrodes 230 can be modulated. This limits the temperature to the interior surface 12 of the vessel 110 so as to accomplish the shrinkage that is needed with minimal trauma or cell damage.

As the contraction of the lumen 14 commences, some fluid may be pulled from the outer balloon 210, thereby allowing the outer balloon 210 to collapse. The removal of fluid could be passive via a check valve or a pressure relief valve (not shown). This would keep the outer surface 205 of the outer balloon 210 smooth as the lumen contracts. In this way, the lumen can be shrunk to the size of the inner balloon 220. The level of the pressure transducers 224 on the inner balloon 220 can be monitored to determine when the outer balloon 210 was in contact with the outer surface 215 of the inner balloon 220. During surgery, this event could signal that the lumen 14 has contracted to the diameter of the inner balloon 220 and that the heating and shrinkage of the vessel 110 could be stopped.

Another feature of use to the surgeon could be one or more markers 255, which may appear in the form of spots, or perhaps lines on the inner and/or outer balloons 220 and 210. The markers 255 are preferably radio opaque. These markers 255 would allow the physician to see where the inner and/or outer balloon surfaces were and help outline the inner surface of the aneurism. Thus, the physician could see in real-time what was happening with regard to the contraction process. Many physicians are working with C-Frame fluoroscopy units, and they can swing these units around and look at the patient from different angles and see what is going on during the surgery. So by modulating the power and watching the shrinkage and looking for how tight the lumen is getting around the balloon, it would be possible to interactively size the interior of the shrunk lumen.

T-type thermocouples can give good temperature resolution within the range of interest. A large number of such thermocouples could be accommodated in a device according to the invention because the leads can be made very thin (e.g., as thin as from approximately 0.0051 inch to 0.0007 inch in diameter). Sensor leads of this type are readily commercially available from the Califormia Fine Wire Company of Grover Beach, Calif., U.S.A.

Membrane type pressure transducers can give good pressure resolution at relatively low pressures. A membrane type transducer, would work like a membrane switch. For example, a little diaphragm would complete a circuit when the pressure exceeded a limit value. Such a membrane transducer could be small in diameter and should be too rigid to be triggered by mere fluid pressure within the balloon. Actual physical contact of surfaces could be the design limit for triggering. These transducers could be located so as to protrude from the outer surface of the inner balloon. When a contact was made, it would deflect the transducer downward in order to make a closed switch. The pressure transducers can also be provided on the outer surface of the expandable elastic balloon to make sure that the outer surface of the outer balloon is in contact with the inside of the aneurism.

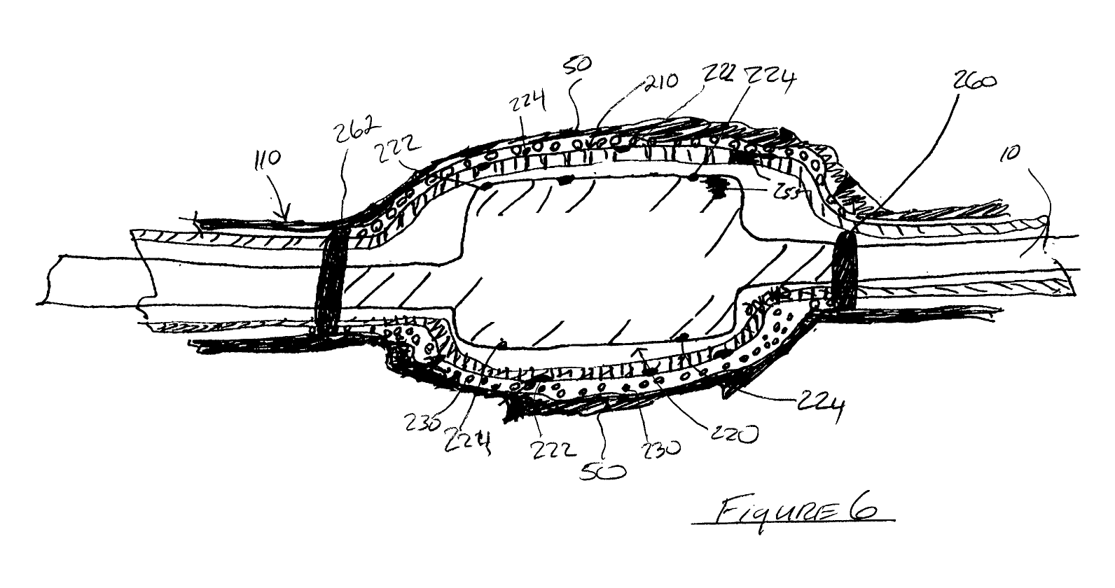

In an embodiment, treatment of an aneurism as described above can be combined with the use of a graft or stent 510. Referring to FIG. 5, an example of a AAA that is a badly extended (a distended aneurism) is shown. A stent 510 could be inserted all the way up to the renal vessels, if it were provided with openings, where the stent could be bonded. Polymer sealing materials and polymer adhesives could be used to bond the stent 510 into place. Another possibility would be a physical clamp. Such a clamp could have teeth that go out into the walls. Those teeth could be hooks of titanium, like a staple. A graft could then extend downward.

There might still be a problem with the aneurism failing because of leakage, particularly in the case of a grossly distended aneurism. Any leakage could still pressurize the aneurism. An RF treatment could be used to pull the lumen wall back to a smaller diameter before installation of the graft. This would toughen and thicken it.

Even after RF treatment, there might still be a problem with the aneurism failing because of leakage, particularly in the case of a grossly distended aneurism that could not be completely shrunk. Any remaining void could be back-filled with a biocompatible material. There are a large number of polymer sealants or collagen type compounds that could be used. The fill material 520 should not be a material that hardens up because the abdominal aorta must be able to bend or fold, and has to be quite compliant. The fill material could be a biocompatible flexible polymer. The fill material could be a collagen compound. The fill material can be nonresorbable, preferably one that can be foamed.

A silicone foam could be used as the fill material depending on the curing temperature. There should not be any agent, catalyst, compound, etc. in the fill material that is not biocompatible. This is to avoid a thrombogenic, or a cytotoxic, or an immune response problem.

In this way, the physician can go in on the outside of the graft and fill what is left of the aneurism, or fill the entire aneurism, if no RF treatment is used. The invention can the shrink and/or stent and/or graft and/or fill, or any possible combination thereof. The goal is to, as much as possible, attain a seal between the fill-in material, the sealant material, and the wall so that blood does not continue to expand the aneurism.

This sealing off would help to prevent restenosis. Restenosis is where there is interluminal growth; it is not necessarily growth of the vessel itself. To help prevent restenosis, it can be advantageous to compound some collagen in with the fill polymer(s), or use a collagen based fill material.

Optionally, the invention can include the provision of drugs in the collagen, or the polymer, or the foam which composes the fill material. These drugs could be for time release and these drugs could be there to block restenosis and/or to curb biologic infection.

The foregoing description of various embodiments of the invention have been presented for purposes of illustration and description. It is not intended to limit the invention to the precise forms disclosed. Many modifications and equivalent arrangements will be apparent.

What is claimed is:

1. An apparatus for treating an aneurysm in a vessel, the apparatus comprising:
a insertion member adapted to be inserted into a lumen of the vessel; and
a first electrode device coupled the insertion member, the first electrode device being positionable in the vessel to apply electrical energy in a radial direction to an interior surface of the vessel, the interior surface forming at least a portion of a surface that circumscribes a cross-section of the vessel.

2. The apparatus of claim 1, wherein the first electrode device is coupled to a power source and applies sufficient electrical energy to heat the interior surface to between approximately 45 to 95 degrees centigrade.

3. The apparatus of claim 1, wherein the first electrode device is coupled to a power source and applies sufficient electrical energy to heat the interior surface to between approximately 60 to 80 degrees centigrade.

4. The apparatus of claim 1, wherein the insertion member is a catheter.

5. The apparatus of claim 1, wherein the first electrode device is coupled to rotate about the insertion member to apply a current to the interior surface.

6. The apparatus of claim 5, where in the rotation of the first electrode device is discrete, and circumscribes the cross-section of the vessel.

7. The apparatus of claim 6, wherein the rotation of the first electrode device is continuous.

8. The apparatus of claim 1, wherein the first electrode device is moveable along the insertion member in a direction defined by a longitudinal axis of the lumen.

9. The apparatus of claim 1, wherein the first electrode device is cylindrical and mounts over the insertion member to apply electrical energy to the interior surface.

10. The apparatus of claim 1, wherein the insertion member is a probe, and the first electrode device is metallic and shoe-shaped, the first electrode device conducting electrical energy to the interior surface.

11. The apparatus of claim 1, wherein the first electrode device applies a radio frequency current to the interior surface.

12. The apparatus of claim 11, wherein a second first electrode device is placed away from the interior surface where the radio frequency current is applied, and wherein an area of the first electrode device is greater than an area of the second first electrode device.

13. The apparatus of claim 12, wherein the second electrode device is coupled to the power source to apply a current to the vessel that is too small to measurably heat a surface of the vessel.

14. The apparatus of claim 13, wherein the power supply produces a voltage difference between the first electrode device and the second electrode device that is approximately 200 volts.

15. The apparatus of claim 1, wherein the first electrode device comprises:
an inner balloon positioned over the insertion member;
an outer balloon positioned over the inner balloon to be proximal to at least a portion of the interior surface;
a plurality of electrodes forming a pattern on an outer surface of the outer balloon, the electrodes applying the electrical energy to the interior surface.

16. The apparatus of claim 15, wherein a plurality of pressure transducers are positioned on an outer surface of the inner balloon.

17. The apparatus of claim 15, wherein individual electrode in the plurality of electrodes are modulated in a sequence to apply the electrical energy to the interior surface.

18. The apparatus of claim 17, wherein the interior surface circumscribes the cross-section of the vessel.

19. The apparatus of claim 1, wherein the first electrode device comprises:
an inelastic inner balloon positioned over the insertion member;
an elastic outer balloon surrounding at least a portion of the inner balloon to be proximal to at least a portion of the interior surface;
a plurality of electrodes forming a pattern on an outer surface of the outer balloon, the electrodes applying the electrical energy to the interior surface.

20. The apparatus of claim 19, wherein the outer balloon is inflatable to contact the interior surface, and the inner balloon is expandable to a predetermined diameter.

21. The apparatus of claim 20, wherein a space between the inner balloon when expanded and the outer balloon when inflated is filled with an electrolyte solution.

22. The apparatus of claim 20, wherein the inner balloon is adapted to receive a fluid to expand to the predetermined fluid.

23. The apparatus of claim 20, wherein a plurality of pressure sensors are located on the outer surface of the outer balloon to ascertain the position of the outer surface of the outer balloon with respect to the interior surface.

24. The apparatus of claim 19, wherein individual electrode in the plurality of electrodes are modulated in a sequence to apply the electrical energy to the interior surface.

25. The apparatus of claim 24, wherein the plurality of electrodes are multiplexed with circuitry for interconnecting individual electrodes in the plurality of electrodes.

26. The apparatus of claim 19, further comprising temperature sensors for determining the temperature of the inner surface.

27. The apparatus of claim 26, wherein a plurality of the temperature sensors are dispersed on the outer surface of the outer balloon to modulate the electrodes, each temperature sensor in the plurality of temperature sensors adapted to monitor a temperature of an area of the inner surface, wherein the electrodes modulate according to the temperature of each area monitored by each temperature sensor in the plurality of temperature sensors.

28. The apparatus of claim 19, wherein the outer balloon expands upon receiving a fluid, and contracts upon releasing the fluid.

29. The apparatus of claim 28, wherein the outer balloon contracts by releasing the fluid via a valve.

30. The apparatus of claim 28, wherein an outer surface of the inner balloon includes a plurality of pressure transducers that detect the outer balloon contacting the inner balloon.

31. The apparatus of claim 19, wherein at least one of the inner balloon and the outer balloon include a radio opaque area for detecting the position of the respective inner balloon or outer balloon with respect to the interior surface.
